(19) United States
(12) Patent Application Publication
(10) Pub. No.: US 2009/0181415 A1
(43) Pub. Date: Jul. 16, 2009

(54) PREDICTION OF GENOTOXICITY

(76) Inventors: Hans Marcus Ludwig Bitter, San Francisco, CA (US); David Michael Goldstein, San Jose, CA (US); Nina Gonzaludo, Palo Alto, CA (US); Stephan Kirchner, Loerrach (DE); Kyle Louis Kolaja, San Mateo, CA (US); Andrew James Olaharski, Sunnyvale, CA (US)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 12/339,499

(22) Filed: Dec. 19, 2008

Related U.S. Application Data
(60) Provisional application No. 61/107,161, filed on Oct. 21, 2008.
(60) Provisional application No. 61/015,291, filed on Dec. 20, 2007.

(51) Int. Cl. C12Q 1/48

(52) U.S. Cl. 435/15

(57) ABSTRACT

The likelihood that a compound will exhibit genotoxicity in a micronucleus test is predicted by the ability of the compound to inhibit a plurality of kinases from a selected group.

This application claims priority from U.S. Ser. No. 61/107,161, filed Oct. 21, 2008 and U.S. Ser. No. 61/015,291, filed Dec. 20, 2007, both incorporated herein by reference in full.

### FIELD OF THE INVENTION

This invention relates generally to the field of toxicology. More particularly, the invention relates to methods for predicting genotoxicity, and methods for screening compounds for potential genotoxicity.

### BACKGROUND OF THE INVENTION

The micronucleus test (“MNT”) is a common assay in the pharmaceutical industry routinely used to detect chromosome damage. A micronucleus forms when whole chromosomes or chromosome fragments do not incorporate into the daughter nuclei following the completion of mitosis. Aneugens and clastogens, chemicals which cause chromosomal loss/gain and breakage, respectively, will cause significant increases in micronuclei formation and can be detected using the assay. Thus, micronuclei are biomarkers of chromosome damage and the micronucleus assay is a sensitive method to detect chemicals which are aneugens and/or clastogens. The micronucleus assay is widely used in the pharmaceutical industry as evidence of genotoxicity (or lack thereof).

However, performing the micronucleus assay is laborious and time consuming, false positive results can occur when testing at cytotoxic doses, and large amounts of supplies (cells, reagents for cell-line maintenance, and compound) are required to perform the assay.

Kinases are enzymes responsible for phosphorylating substrates and disseminating inter- and intracellular signals, including the initiation, propagation, and termination of chromosome replication during mitosis. Kinases are often targeted for inhibition by pharmaceutical companies because many signaling cascades have known roles in a variety of diseases. Small molecule kinase inhibitors (SMKIs) often are developed to competitively bind to the kinase ATP binding pocket, blocking the ability of the enzyme to phosphorylate substrates. SMKIs often inhibit many kinases in addition to the desired target due to the highly conserved nature of the ATP binding pocket within the kinome, thus toxicities associated with off-target kinase inhibition is a concern for this pharmaceutical class of compounds. In particular, post-metaphase genetic toxicity, manifested as positive micronucleus results, is a common toxicological liability for SMKIs.

### SUMMARY OF THE INVENTION

We have now invented a method for predicting which compounds will demonstrate positive (i.e., genotoxic) results in a micronucleus assay, using a method that is faster, uses smaller quantities of reagents, and is easily automated.

One aspect of the invention is a method for predicting the genotoxicity of a compound, the method comprising providing a test compound; and determining the ability of the compound to inhibit the kinase activity of a number of kinases selected from the group of primary kinases consisting of CAMK2A, CAMK2D, DYRK1B, MAPK15, PCTK2, PFTK1, PCTK1, PCTK3, CDK2, GSK3A, CDK3, CLK2, MELK, BRSK2, CAMK1, STK3, MYLK, CDK5, FLT3, FLT3.ITD, PRKR, and AMPKα2, wherein inhibition of at least twelve of the 22 kinases by at least 50% indicates a likelihood that said test compound will demonstrate genotoxicity. If at least 12 of the 22 primary kinases are inhibited by 100%, this strongly and reliably indicates that the test compound would test as toxic in the MNT assay. Another aspect of the invention comprises the method wherein the group of kinases further comprises one or more kinases selected from the group of secondary kinases consisting of SLK, NUAK1, CAMKK2, BRSK1, GSK3B, TTK, CAMK2G, ALK, AAK1, ACVR2A, CLK1, BIKE, SNARK, LIMK2, PIP5K1A, STK16, LIMK1, DAPK1, PTK2B, CDK9, RPS6KA1.Kin.Dom.1, and CLK4.

Another aspect of the invention is the method for screening candidate compounds for potential genotoxicity, comprising providing a plurality of compounds; and determining the ability of each compound to inhibit the kinase activity of a number of kinases selected from the group consisting of CAMK2A, CAMK2D, DYRK1B, MAPK15, PCTK2, PFTK1, PCTK1, PCTK3, CDK2, GSK3A, CDK3, CLK2, MELK, BRSK2, CAMK1, STK3, MYLK, CDK5, FLT3, FLT3.ITD, PRKR, and AMPKα2, wherein inhibition of at least five of said kinases by 100% indicates a likelihood that said test compound will demonstrate genotoxicity. Another aspect of the invention comprises the method wherein the group of kinases further comprises the group consisting of SLK, NUAK1, CAMKK2, BRSK1, GSK3B, TTK, CAMK2G, ALK, AAK1, ACVR2A, CLK1, BIKE, SNARK, LIMK2, PIP5K1A, STK16, LIMK1, DAPK1, PTK2B, CDK9, RPS6KA1.Kin.Dom.1, and CLK4.

One aspect of the invention is a method for predicting the genotoxicity of a compound, the method comprising providing a test compound; and determining the ability of the compound to inhibit the kinase activity of a number of kinases selected from the group consisting of CDK2, CLK1, DYRK1B, ERK8, GSK3A, GSK3B, PCTK1, PCTK2, STK16, TTK, CLK2, ERK3, and PRKR, or the group consisting of CDK2, CLK1, DYRK1B, ERK8 (MAPK15), GSK3A, GSK3B, PCTK1, PCTK2, STK16, TTK, CDK17, CLK4, and PCTK3, wherein inhibition of at least five of said kinases by 100% indicates a likelihood that said test compound will demonstrate genotoxicity.

Another aspect of the invention is the method for screening candidate compounds for potential genotoxicity, comprising providing a plurality of compounds; and determining the ability of each compound to inhibit the kinase activity of a number of kinases selected from the group consisting of CDK2, CLK1, DYRK1B, ERK8, GSK3A, GSK3B, PCTK1, PCTK2, STK16, TTK, CLK2, ERK3, and PRKR, or the alternate group consisting of CDK2, CLK1, DYRK1B, ERK8 (MAPK15), GSK3A, GSK3B, PCTK1, PCTK2, STK16, TTK, CDK7, CLK4, and PCTK3, wherein inhibition or specific binding of at least five of said kinases by 100% indicates a likelihood that said test compound will demonstrate genotoxicity.

### DETAILED DESCRIPTION OF THE INVENTION

### Definitions

Unless otherwise stated, the following terms used in this application, including the specification and claims, have the definitions given below. It must be noted that, as used in the specification and the appended claims, the singular forms “a”, “an,” and “the” include plural referents unless the context clearly dictates otherwise.

The term “genotoxicity” as used herein refers to compounds that produce chromosomal aberrations, including breakage (clastogens) or abnormal copy number (aneugens). In this context, “genotoxicity” refers to a positive result in a micronucleus test. A “likelihood of genotoxicity” means specifically that the compound in question is predicted to demonstrate genotoxicity in an MNT with at least 75% confidence.

The term “test compound” refers to a substance which is to be tested for genotoxicity. The test compound can be a candidate drug or lead compound, a chemical intermediate, environmental pollutant, a mixture of compounds, and the like.

The term “kinase” refers to an enzyme capable of attaching and/or removing a phosphate group from a protein or molecule. “Inhibition of kinase activity” refers to the ability of a compound to reduce or interfere with such phosphatase activity. As binding affinity of a small molecule for a given kinase correlates well with the ability of said molecule to inhibit the kinase activity, binding affinity is considered synonymous with kinase activity herein, and high binding affinity is considered equivalent to high kinase inhibitory activity. The correlation between binding affinity and kinase inhibition is described by M. A. Fabian et al., Nature Biotechnol (2005) 23:329-36, incorporated herein by reference in full.

The term “primary kinases” refers to the following set of kinases (also identified by accession number in parentheses): CAMK2A (NP_{—}741960.1), CAMK2D (AAD20442.1), DYRK1B (NP_{—}004705.1), MAPK15 (NP_{—}620590.2), PCTK2 (CAA47004.1), PFTK1 (NP_{—}036527.1), PCTK1 (NP_{—}006192.1), PCTK3 (NP_{—}002587.2), CDK2 (cyclin dependent kinase 2, NP_{—}001789.2), GSK3A (NP_{—}063937.2), CDK3 (NP_{—}001249.1), CLK2 (NP_{—}003984.2), MELK (NP_{—}055606.1), BRSK2 (NP_{—}003948.2), CAMK1 (NP_{—}003647.1), STK3 (NP_{—}006272.1), MYLK (NP_{—}444254.3), CDK5 (NP_{—}004926.1), FLT3 (NP_{—}004110.2), FLT3.ITD (NP_{—}004110.2), PRKR (NP_{—}002750.1), and AMPKα2 (NP_{—}006243.2). The term “secondary kinases” refers to the following set: SLK (NP_{—}055535.2), NUAK1 (NP_{—}055655.1), CAMKK2 (NP_{—}006540.3), BRSK1 (NP_{—}115806.1), GSK3B (NP_{—}002084.2), TTK (NP_{—}003309.2), CAMK2G (NP_{—}751913.1), ALK (NP_{—}004295.2), AAK1 (NP_{—}055726.3), ACVR2A (NP_{—}001607.1), CLK1 (AAA61480.1), BIKE (NP_{—}060063.2), SNARK (NP_{—}112214.1), LIMK2 (NP_{—}005560.1), PIP5K1A (AAC50911.1), STK16 (CAA06700.1), LIMK1 (NP_{—}002305.1), DAPK1 (NP_{—}004929.2), PTK2B (NP_{—}775267.1), CDK9 (NP_{—}001252.1), RPS6KA1.Kin.Dom.1 (NP_{—}002944.2), and CLK4 (NP_{—}065717.1). The term “identified kinases” refers to the following set of kinases (also identified by accession number): CDK2 (NM_{—}001798.2), CLK1 (NM_{—}004071.1), DYRK1B (NP_{—}004705.1), ERK8 (aka MAPK15, NP_{—}620590.2), GSK3A (D63424.1), GSK3B (NP_{—}002084.2), PCTK1 (NM_{—}006201.2), PCTK2 (CAA47004.1), STK16 ( )NM_{—}003691.1, TTK (NM_{—}003318.2), CLK2 (NM_{—}003993.1), ERK3 (NP_{—}002739.1), and PRKR (NM_{—}002759.1). “Alternate identified kinases” refers to the set of kinases consisting of CDK2, CLK1, DYRK1B, ERK8 (MAPK15), GSK3A, GSK3B, PCTK1, PCTK2, STK16, TTK, CDK7, CLK4, and PCTK3.

All patents and publications identified herein are incorporated herein by reference in their entirety.

### General Method

The invention provides a method for quickly determining the likelihood that a given compound will exhibit genotoxicity in an MNT assay by examining the interaction between the compound and a number of kinases (kinase binding and/or inhibition). As kinase inhibition and/or binding can be determined quickly, and by using automated methods, the method of the invention enables high-throughput screening of compounds for genotoxicity (or lack thereof).

Thus, one aspect of the invention is a method for predicting the genotoxicity of a compound, said method comprising providing a test compound; determining the ability of the compound to inhibit the kinase activity of at least ten kinases selected from the group consisting of CDK2, CLK1, DYRK1B, ERK8, GSK3A, GSK3B, PCTK1, PCTK2, STK16, TTK, CLK2, ERK3, and PRKR, wherein inhibition of at least five of said kinases by 100% indicates a likelihood that said test compound will demonstrate genotoxicity.

Another aspect of the invention is the method described above, wherein the second step further comprises determining the ability of the compound to inhibit the kinase activity of at least one kinase selected from the group consisting of MKNK2, SgK085, PIM2, TNNI3K, KIT, MELK, AURKA, CLK3, AAK1, DCAMKL3, LIMK1, FLT1, MAP2K4, PIM3, AURKB, ERK2, CSNK1A1L, DAPK3, MLCK, CLK3, PFTK1, PRKD3, AURKC, ERK5, STK17A, MST4, CDK3, MYLK, CDC2L1, QIK, CDK11, PLK1, PDGFRβ, PRKCM, MAPK4, PIP5K2B, CSNK1D, RPS6KA1.Kin.Dom.1, CDK5, PLK3, BIKE, PLK4, CAMK2A, STK3, CSNK2A1, STK17B, CDK8, MAP2K6, PIM1, MAP2K3, CDK7, IKKε, TGFBR2, CDK9, CLK4, and PCTK3.

Another aspect of the invention is the method wherein the test compound is tested at a concentration of about 10 μM. Another aspect of the invention is the method wherein the second step comprises determining the ability of the compound to inhibit the kinase activity of at least twelve kinases selected from the identified group. Another aspect of the invention is the method wherein the second step comprises determining the ability of the compound to inhibit the kinase activity of all kinases in the group.

Another aspect of the invention is a method for predicting the genotoxicity of a compound, by providing a test compound; and determining the ability of the compound to inhibit the kinase activity of at least ten kinases selected from the group consisting of CDK2, CLK1, DYRK1B, ERK8 (MAPK15), GSK3A, GSK3B, PCTK1, PCTK2, STK16, TTK, CDK7, CLK4, and PCTK3, wherein inhibition of at least five of said kinases by 100% indicates a likelihood that the test compound will demonstrate genotoxicity.

Another aspect of the invention is the method wherein the second step further comprises determining the ability of the compound to inhibit the kinase activity of at least one kinase selected from the group consisting of MKNK2, SgK085, PIM2, TNNI3K, KIT, MELK, AURKA, CLK3, AAK1, DCAMKL3, LIMK1, FLT1, MAP2K4, PIM3, AURKB, ERK2, CSNK1A1L, DAPK3, MLCK, CLK3, PFTK1, PRKD3, AURKC, ERK5, STK17A, MST4, CDK3, MYLK, CDC2L1, QIK, CDK11, PLK1, PDGFRβ, PRKCM, MAPK4, PIP5K2B, CSNK1D, RPS6KA1.Kin.Dom.1, CDK5, PLK3, BIKE, PLK4, CAMK2A, STK3, CSNK2A1, STK17B, CDK8, MAP2K6, PIM1, MAP2K3, CDK7, IKKε, TGFBR2, CDK9, CLK4, and PCTK3.

Another aspect of the invention is the method wherein the test compound is tested at a concentration of about 10 μM.

Another aspect of the invention is the method wherein the second step comprises determining the ability of the compound to inhibit the kinase activity of at least twelve kinases selected from the group.

Another aspect of the invention is the method wherein the second step comprises determining the ability of the compound to inhibit the kinase activity of all kinases in the group. Another aspect of the invention is a method for screening compounds for potential genotoxicity, comprising: providing a plurality of test compounds; and determining the ability of each compound to inhibit the kinase activity of at least ten kinases selected from the group consisting of CDK2, CLK1, DYRK1B, ERK8, GSK3A, GSK3B, PCTK1, PCTK2, STK16, TTK, CLK2, ERK3, and PRKR, or the alternate group consisting of CDK2, CLK1, DYRK1B, ERK8 (MAPK15), GSK3A, GSK3B, PCTK1, PCTK2, STK16, TTK, CDK7, CLK4, and PCTK3; where inhibition of at least five of said kinases by 100% indicates a likelihood that said test compound will demonstrate genotoxicity.

Another aspect of the invention is the method further comprising rejecting compounds that demonstrate a likelihood of genotoxicity.

Another aspect of the invention is the method wherein the ability of the compound to inhibit the kinase activity is determined by measuring the binding affinity of the compound for said kinases.

Another aspect of the invention is a test substrate, comprising: a solid support; and immobilized on said solid support, the kinases CDK2, CLK1, DYRK1B, ERK8, GSK3A, GSK3B, PCTK1, PCTK2, STK16, TTK, CLK2, ERK3, and PRKR or the kinases CDK2, CLK1, DYRK1B, ERK8 (MAPK15), GSK3A, GSK3B, PCTK1, PCTK2, STK16, TTK, CDK7, CLK4, and PCTK3. Another aspect of the invention is the test substrate of claim 14, further comprising immobilized on said solid support, a kinase selected from the group consisting of MKNK2, SgK085, PIM2, TNNI3K, KIT, MELK, AURKA, CLK3, AAK1, DCAMKL3, LIMK1, FLT1, MAP2K4, PIM3, AURKB, ERK2, CSNK1A1L, DAPK3, MLCK, CLK3, PFTK1, PRKD3, AURKC, ERK5, STK17A, MST4, CDK3, MYLK, CDC2L1, QIK, CDK1, PLK1, PDGFRβ, PRKCM, MAPK4, PIP5K2B, CSNK1D, RPS6KA1.KD1, CDK5, PLK3, BIKE, PLK4, CAMK2A, STK3, CSNK2A1, STK17B, CDK8, MAP2K6, PIM1, MAP2K3, CDK7, IKKε, TGFBR2, CDK9, CLK4, and PCTK3.

In practice, binding and inhibition can be determined using methods known in the art. See, for example, M. A. Fabian et al., Nature Biotechnol (2005) 23:329-36, incorporated herein by reference in full. In general, the binding affinity of a compound for a given kinase correlates well with the ability of the compound to inhibit the activity of that kinase, so that binding affinity is a reliable substitute for inhibitory activity. Binding affinity may be determined by a variety of methods known in the art; for example by competitive assay using an immobilized kinase (or an immobilized test compound, or an immobilized competing ligand, any of which may be labeled). Compounds and kinases can be immobilized by standard methods, for example by biotinylation and capture on a streptavidin-coated substrate.

Thus, one can prepare a test substrate having, for example, a plurality of immobilized kinases, preferably comprising a plurality the primary kinases or identified kinases. In one embodiment, the substrate comprises all of the primary kinases. In another embodiment, the substrate further comprises a plurality of the secondary kinases. In another embodiment, the substrate comprises all of the primary and secondary kinases. In another embodiment, the substrate comprises all of the identified kinases. In another embodiment, the substrate further comprises a plurality of the alternate identified kinases. In another embodiment, the substrate comprises all of the identified kinases and the secondary kinases. The kinases can be immobilized directly (i.e., by adsorption, covalent bond, or biotin-avidin binding or the like) to the surface, or indirectly (for example by binding to a ligand that is tethered to the surface by adsorption, covalent bond, biotin-avidin or other linkage). The kinases are then contacted with the test compound(s), and the affinity (or enzyme inhibition) determined, for example by measuring the binding of labeled compound or loss of labeled competitor.

The kinase affinity of each compound is measured against at least ten of the 22 primary or 13 identified kinases. Use of a larger number of kinases selected from these sets results in a prediction of genotoxicity with higher confidence. A compound with high total activity (for example, demonstrating high affinity for at least five of the primary or identified kinases, preferably eight or more) has a high likelihood of genotoxicity: this compound is predicted to test positive for genotoxicity in the MNT. A compound having low total activity (for example, showing only low affinity for the selected kinases, or showing high affinity to only 1-4 selected kinases) is predicted to test negative in the MNT.

Candidate drugs that test positive in the assay of the invention (i.e., that are predicted to demonstrate genotoxicity in the MNT) are generally identified as “genotoxic” or “potentially genotoxic”, and rejected or otherwise dropped from further development. In the case of high-throughput screening applications, such compounds can be flagged as toxic (for example, by the software managing the system in the case of an automated high-throughput system), thus enabling earlier decision making.

Thus, one can use the method of the invention to prioritize and select candidate compounds for pharmaceutical development based in part on the potential of the compound for genotoxicity. For example, if one has prepared a plurality of compounds (e.g., 50 or more), having similar activity against a selected target, and desires to prioritize or select a subset of said compounds for further development, one can test the entire group of compounds in the method of the invention and discard or reject all those compounds that exhibit positive signs of genotoxicity. This reduces the cost of pharmaceutical development, and the amount invested in any compound selected for development by identifying an important source of toxicity early on. Because the method of the invention is fast and easily automated, it enables the bulk screening of compounds that would otherwise not be possible or practical.

Environmental pollutants and the like can also be identified using the method of the invention, in which case such compounds are typically identified for further study into their toxic properties. In this application of the method of the invention, one can fractionate an environmental sample (for example, soil, water, or air, suspected of contamination) by known methods (for example chromatography), and subject said fractions to the method of the invention. Fractions that display signs of genotoxicity can then be further fractionated, and (using the method of the invention), the responsible toxic agents identified. Alternatively, one can perform the method of the invention using pure or purified compounds that are suspected of being environmental pollutants to determine their potential for genotoxicity. Because the method of the invention is fast and easily automated, it enables the bulk screening of samples that would otherwise not be possible or practical.

The following additional kinases can also be tested: high affinity of a compound for one or more of these additional kinases (in addition to a majority of the primary or identified kinases) correlates with a higher likelihood of genotoxicity. The additional kinases (and accession numbers) are: MKNK2 (NM_{—}017572.1), SgK085 (NP_{—}001012418.1), PIM2 (NM_{—}006875.1), TNNI3K (NM_{—}015978.1), KIT (NM_{—}000222), MELK (NM_{—}014791.1), AURKA (NM_{—}003600.1), CLK3 (NM_{—}003992.1), AAK1 (NM_{—}014911.1), DCAMKL3 (XP_{—}047355.6), LIMK1 (NM_{—}002314.2), FLT1 (NM_{—}002019.2), MAP2K4 (NP_{—}003001.1), PIM3 (NP_{—}001001852.1), AURKB (NM_{—}004217.1), ERK2 (NM_{—}138957.1), CSNK1A1L (NM_{—}145203.1), DAPK3 (NM_{—}001348.1), MLCK (NP_{—}872299.1), CLK3 (NM_{—}003992.1), PFTK1 (NP_{—}036527.1), PRKD3 (NP_{—}005804.1), AURKC (NM_{—}003160.1), ERK5 (NP_{—}002740.2), STK17A (NM_{—}004760.1), MST4 (NM_{—}016542.2), CDK3 (NP_{—}001249.1), MYLK (NP_{—}444254.3), CDC2L11(NP_{—}277023.1), QIK (XM_{—}041314.4), CDK11 (NP_{—}055891.1), PLK1 (NM_{—}005030.2), PDGFRβ (NM_{—}002609.2), PRKCM (NM_{—}002742.1), MAPK4 (NP_{—}002738.2), PIP5K2B (NP_{—}003550.1), CSNK1D (NM_{—}001893.3), RPS6KA1 (KD1) (NM_{—}002953.3), CDK5 (NP_{—}031694.1), PLK3 (NM_{—}004073.1), BIKE (NM_{—}017593.2), PLK4 (NM_{—}014264.2), CAMK2A (NM_{—}015981.1), STK3 (NP_{—}006272.2), CSNK2A1 (NM_{—}001895.1), STK17B (NM_{—}004226.1), CDK8 (NP_{—}001251.1), MAP2K6 (NM_{—}002758.3), PIM1 (NM_{—}002648.1), MAP2K3 (NP_{—}002747.2), CDK7 (NP_{—}001790.1), IKKε (NP_{—}054721.1), TGFBR2 (NM_{—}003242.4), CDK9 (NP_{—}001252.1), CLK4 (NM_{—}020666.1), and PCTK3 (NP_{—}002587.2).

### EXAMPLES

### Example 1

To identify the set of kinases that would indicate a likelihood that a test compound would demonstrate genotoxicity, the following analysis was carried out. First, 54 suitable small molecule kinase inhibitors (“SMKIs”) were selected to form a training set. Second, for each compound in the training set, an in vitro MNT result and single point inhibition profiles against 290 kinases were acquired. A statistical analysis was then performed to (1) build a model using said single point kinase inhibition profiles to predict said MNT result and (2) identify the kinases correlated with MNT results. Finally, the model was validated against an additional set of 33 SMKIs not used for training.

The in vitro micronucleus assay has been described in detail previously (M. Fenech, Mutation Res (2000) 455(1-2):81-95). The established permanent mouse lymphoma cell line L5178Y tk^{+/-} (ATCC CRL 9518), growing in suspension, was used for this experiment. In general, compounds were tested up to 500 μg/mL, and at least 12 concentration levels were tested. The top dose for evaluation was generally selected to observe acceptable toxicity (decrease of the relative cell count (RCC) below 50%) or clear signs of precipitation in the aqueous medium. If the compound was soluble and non-toxic, a maximal dose level of 5000 μg/mL was set. For assessment of cytotoxicity, relative cell counts (RCC, as % negative control) were calculated. Slides were prepared by setting the cell density to approximately 1×10⁶ cells/mL and centrifuging onto clean glass slides using a cytospin (1000 rpm, 5 min). Fixation of cells and storage was performed in ice cold methanol (−20° C., at least 4 h). Slides were incubated for 5 min with H 33258 (1 μg/mL PBS/CMF) and mounted with 10 μL antifade for fluorescence microscopy. A minimum of 3 concentration levels were analysed for the presence of micro-nucleated cells with the aid of an epifluorescence microscope equipped with appropriate filter sets. A compound is considered to possess clastogenic/aneugenic activity if one or more concentrations show at least a 2-fold increase in the number of micronucleated cells in comparison to the concurrent negative control.

Fifty-four compounds were selected for inclusion in the training set, based on a number of criteria including selective kinase inhibition profiles, minimization of redundancy, and chemical diversity. From an internal database of SMKIs, only compounds that had selective kinase inhibition profiles were considered, where a selective compound was considered to be one that inhibited six or fewer kinases at single point inhibition values greater than 95%, and eleven or fewer kinases at values greater than 85%. Kinase inhibition was determined using the method set forth in M. A. Fabian et al., Nature Biotechnol (2005) 23:329-36. In cases where a number of compounds were selective against many of the same kinases, only one of the compounds was selected, to minimize redundancy or over-representation of those kinases. After these filtering steps, a chemically diverse set was selected based on physical properties, including A Log P, molecular weight, number of hydrogen donors and acceptors, number of rotatable bonds, number of atoms, number of rings, number of aromatic rings, and number of fragments. Diversity was defined using the “Diverse Molecules” filter, based on a maximum dissimilarity method, in SciTegic's Pipeline Pilot 6.0.2.

Inhibition profiles against 290 kinases and in vitro MNT results were acquired for each compound in the training set (N=54). Three different readouts were obtained for the MNT results: negative (N=22), positive (N=26), and weakly positive (N=6). The six weakly positive were assigned to either negative or positive labels based on the % MN cells at the concentration at which the inhibition profiles were performed. This led to five of the six compounds being re-assigned as negative, giving a total of 27 negative and 27 positive compounds.

Pre-processing was first performed across the set of all inhibition profiles to remove uninformative or biased kinases. Kinases with no variance across the set of 54 compounds were removed, as they were not informative. JNK and p38 isoforms were removed to reduce the bias of the large number of compounds in the training set that were developed to target those kinases. To ensure that the removal of JNK and p38 isoforms did not introduce a different form of bias, we performed an additional analysis whereby we considered only those training set compounds not developed for these kinase targets, and found that none of the JNK and p38 isoforms were correlated with MNT results.

Feature selection (FS) and pattern recognition (PR) were performed in several phases in order to build the model. For all analyses, cross validation was used to assess the model performance over several trials. Each trial randomly split the initial data into a training set and a test set; the training set was used to build the temporary model, and the test set was used to predict results and then verify performance. Feature selection methods were used to determine which kinases, or “features”, were likely to correlate most with MNT result. In each trial, the inhibition values against the features chosen were used as input for a pattern recognition method, which then predicted the positive or negative result.

In the first phase, feature selection methods were divided into two groups: methods that could handle a large input data set (FS1), and methods that performed better with less data (FS2). Different combinations of FS1, FS2, and PR were tested over several trials using 10 five-fold cross-validations. The combination of methods with the lowest mean error rate was chosen for the next phase of the analysis. This combination includes a Kolmogorov-Smirnov/T-test hybrid algorithm for FS1, Random Forests for FS2, and Support Vector Machines for PR (T. Hastie et al., “The Elements of Statistical Learning” (2001, Springer-Verlag); R. O. Duda et al., “Pattern Classification, 2^{nd} Ed.” (2000, Wiley-Interscience); and “Feature Extraction—Foundations and Applications” (2006, Springer-Verlag, I. Guyon et al. Eds.)).

The chosen combination of methods from the first phase were tuned for optimal performance. Several parameters were optimized, including the number of kinases to be used in the model. The tuning process showed that within several trials, the mean error rate was lowest when the number of kinases chosen as significant after FS1 and FS2 was 13. Thus the model was adjusted with the optimal parameters, then specified to choose the 13 most significant features as input for PR.

The accuracy of the model using this combination of feature selection and pattern recognition methods, number of features, and optimal tuning parameters was then assessed by performing 50 five-fold cross-validations. Importantly, the feature selection and pattern recognition was performed within each cross-validation fold. The resulting model had an accuracy of 80%±4%: that is, the model on average correctly predicted MNT results 80% of the time.

The 50 five-fold cross-validations were also used to determine the kinases correlated with MNT result. The selection of kinases was based on the number of times a kinase was chosen as significant amongst the 250 trials (50 five-fold cross-validations). 55 out of the original 290 kinases were chosen at least once as significant. Those kinases that were chosen with a frequency of greater than 50% (N=13) were selected to be included in the final model. Over multiple runs of testing, the kinase inhibition profiles against these 13 kinases were found to be significant in predicting actual MNT result at least 50% of the time. That is, SMKIs with a positive in vitro MNT result tended to have high levels of inhibition against the thirteen kinases.

For each SMKI, the model consists of single point kinase inhibition profiles against the following 13 kinases: CDK2, CLK1, DYRK1B, ERK8 (MAPK15), GSK3A, GSK3B, PCTK1, PCTK2, STK16, TTK, CLK2, ERK3, and PRKR. Additionally, an in vitro MNT assay result at the concentration in which the kinase screen was performed is included. A second model based upon quantitative binding constants consisted a second (overlapping) set of thirteen kinases: CDK2, CLK1, DYRK1B, ERK8 (MAPK15), GSK3A, GSK3B, PCTK1, PCTK2, STK16, TTK, CDK7, CLK4, and PCTK3. The kinases selected for the two models are highly similar, demonstrating the robustness of the single point kinase inhibition model.

To assess the utility of the final model, an additional set of 33 compounds were used as a validation set. These 33 compounds were not included in the initial set of 54, but each compound included a single point inhibition value against the thirteen model kinases, plus an in vitro MNT result. Given the validation data, the model was able to accurately predict the MNT result of all compounds, and thus performed with an accuracy of 76%, which lies within our estimated accuracy of the model based on cross-validation.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

### Example 2

Proceeding as described in Example 1 above, but employing an expanded set of training compounds (113 compounds instead of 54), the primary and secondary kinases were identified as more accurately predicting a positive (toxic) result in the MNT assay. The primary kinases identified are (accession number in parentheses): CAMK2A (NP_{—}741960.1), CAMK2D (AAD20442.1), DYRK1B (NP_{—}004705.1), MAPK15 (NP_{—}620590.2), PCTK2 (CAA47004.1), PFTK1 (NP_{—}036527.1), PCTK1 (NP_{—}006192.1), PCTK3 (NP_{—}002587.2), CDK2 (cyclin dependent kinase 2, NP_{—}001789.2), GSK3A (NP_{—}063937.2), CDK3 (NP_{—}001249.1), CLK2 (NP_{—}003984.2), MELK (NP_{—}055606.1), BRSK2 (NP_{—}003948.2), CAMK1 (NP_{—}003647.1), STK3 (NP_{—}006272.1), MYLK (NP_{—}444254.3), CDK5 (NP_{—}004926.1), FLT3 (NP_{—}004110.2), FLT3.ITD (NP_{—}004110.2), PRKR (NP_{—}002750.1), and AMPKα2 (NP_{—}006243.2). The secondary kinases identified are: SLK (NP_{—}055535.2), NUAK1 (NP_{—}055655.1), CAMKK2 (NP_{—}006540.3), BRSK1 (NP_{—}115806.1), GSK3B (NP_{—}002084.2), TTK (NP_{—}003309.2), CAMK2G (NP_{—}751913.1), ALK (NP_{—}004295.2), AAK1 (NP_{—}055726.3), ACVR2A (NP_{—}001607.1), CLK1 (AAA61480.1), BIKE (NP_{—}060063.2), SNARK (NP_{—}112214.1), LIMK2 (NP_{—}005560.1), PIP5K1A (AAC50911.1), STK16 (CAA06700.1), LIMK1 (NP_{—}002305.1), DAPK1 (NP_{—}004929.2), PTK2B (NP_{—}775267.1), CDK9 (NP_{—}001252.1), RPS6KA1.Kin.Dom.1 (NP_{—}002944.2), and CLK4 (NP_{—}065717.1).

Proceeding as described in Example 1 above, 113 small molecule kinase inhibitors were screened for their ability to inhibit 290 kinases. The model was developed as set forth in Example 1 above, except that micronucleus results were based upon concentration, such that positive micronucleus results occurring at concentrations above 10 μM were reclassified as negative, while results that were positive below that threshold were classified as positive. Thirty of the 113 small molecule kinase inhibitors were classified as positive, whereas 83 were negative. All negative classifications were independent of concentration. Instead of using 250 trials (50 five-fold cross-validations), 500 trials were used.

This resulted in identification of 22 primary kinases, inhibition of which correlated strongly with positive (toxic) MNT results. The primary kinases identified were CAMK2A, CAMK2D, DYRK1B, MAPK15, PCTK2, PFTK1, PCTK1, PCTK3, CDK2, GSK3A, CDK3, CLK2, MELK, BRSK2, CAMK1, STK3, MYLK, CDK5, FLT3, FLT3.ITD, PRKR, and AMPKα2. If a test compound exhibits inhibition of about 100% against at least 12 of the 22 primary kinases, this model predicts that it will exhibit a positive (toxic) response in the MNT assay. The likelihood of a positive MNT response correlates with the number of kinases inhibited, and the degree to which they are inhibited.

In addition, a further group of 22 secondary kinases was identified, inhibition of which (in conjunction with one or more primary kinases) correlates strongly with positive MNT results. The secondary kinases identified were SLK, NUAK1, CAMKK2, BRSK1, GSK3B, TTK, CAMK2G, ALK, AAK1, ACVR2A, CLK1, BIKE, SNARK, LIMK2, PIP5K1A, STK16, LIMK1, DAPK1, PTK2B, CDK9, RPS6KA1.Kin.Dom.1, and CLK4. Where a test compound exhibits inhibition of the primary kinases, inhibition of several secondary kinases further increases the probability of a positive MNT result.

What is claimed:

1. A method for predicting the genotoxicity of a compound, said method comprising:
a) providing a test compound;
b) determining the ability of the compound to inhibit the kinase activity of at least ten kinases selected from the group consisting of CAMK2A (NP_{—}741960.1), CAMK2D (AAD20442.1), DYRK1B (NP_{—}004705.1), MAPK15 (NP_{—}620590.2), PCTK2 (CAA47004.1), PFTK1 (NP_{—}036527.1), PCTK1 (NP_{—}006192.1), PCTK3 (NP_{—}002587.2), CDK2 (NP_{—}001789.2), GSK3A (NP_{—}063937.2), CDK3 (NP_{—}001249.1), CLK2 (NP_{—}003984.2), MELK (NP_{—}055606.1), BRSK2 (NP_{—}003948.2), CAMK1 (NP_{—}003647.1), STK3 (NP_{—}006272.1), MYLK (NP_{—}444254.3), CDK5 (NP_{—}004926.1), FLT3 (NP_{—}004110.2), FLT3.ITD (NP_{—}004110.2), PRKR (NP_{—}002750.1), and AMPKα2 (NP_{—}006243.2), wherein inhibition of at least five of said kinases by 100% indicates a likelihood that said test compound will demonstrate genotoxicity.

2. The method of claim 1, wherein step b) further comprises determining the ability of the compound to inhibit the kinase activity of at least one kinase selected from the group consisting of SLK (NP_{—}055535.2), NUAK1 (NP_{—}055655.1), CAMKK2 (NP_{—}006540.3), BRSK1 (NP_{—}115806.1), GSK3B (NP_{—}002084.2), TTK (NP_{—}003309.2), CAMK2G (NP_{—}751913.1), ALK (NP_{—}004295.2), AAK1 (NP_{—}055726.3), ACVR2A (NP_{—}001607.1), CLK1 (AAA61480.1), BIKE (NP_{—}060063.2), SNARK (NP_{—}112214.1), LIMK2 (NP_{—}005560.1), PIP5K1A (AAC50911.1), STK16 (CAA06700.1), LIMK1 (NP_{—}002305.1), DAPK1 (NP_{—}004929.2), PTK2B (NP_{—}775267.1), CDK9 (NP_{—}001252.1), RPS6KA1.Kin.Dom.1 (NP_{—}002944.2), and CLK4 (NP_{—}065717.1).

3. The method of claim 1, wherein said test compound is tested at a concentration of about 10 μM.

4. The method of claim 1, wherein step b) comprises determining the ability of the compound to inhibit the kinase activity of at least twelve kinases selected from said group.

5. The method of claim 3, wherein step b) comprises determining the ability of the compound to inhibit the kinase activity of all kinases in said group.

6. A method for predicting the genotoxicity of a compound, said method comprising:
a) providing a test compound;
b) determining the ability of the compound to inhibit the kinase activity of at least ten kinases selected from the group consisting of CAMK2A (NP_{—}741960.1), CAMK2D (AAD20442.1), DYRK1B (NP_{—}004705.1), MAPK15 (NP_{—}620590.2), PCTK2 (CAA47004.1), PFTK1 (NP_{—}036527.1), PCTK1 (NP_{—}006192.1), PCTK3 (NP_{—}002587.2), CDK2 (cyclin dependent kinase 2, NP_{—}001789.2), GSK3A (NP_{—}063937.2), CDK3 (NP_{—}001249.1), CLK2 (NP_{—}003984.2), MELK (NP_{—}055606.1), BRSK2 (NP_{—}003948.2), CAMK1 (NP_{—}003647.1), STK3 (NP_{—}006272.1), MYLK (NP_{—}444254.3), CDK5 (NP_{—}004926.1), FLT3 (NP_{—}004110.2), FLT3.ITD (NP_{—}004110.2), PRKR (NP_{—}002750.1), and AMPKα2 (NP_{—}006243.2), wherein inhibition of at least five of said kinases by 100% indicates a likelihood that said test compound will demonstrate genotoxicity.

7. The method of claim 6, wherein step b) further comprises determining the ability of the compound to inhibit the kinase activity of at least one kinase selected from the group consisting of SLK (NP_{—}055535.2), NUAK1 (NP_{—}055655.1), CAMKK2 (NP_{—}006540.3), BRSK1 (NP_{—}115806.1), GSK3B (NP_{—}002084.2), TTK (NP_{—}003309.2), CAMK2G (NP_{—}751913.1), ALK (NP_{—}004295.2), AAK1 (NP_{—}055726.3), ACVR2A (NP_{—}001607.1), CLK1 (AAA61480.1), BIKE (NP_{—}060063.2), SNARK (NP_{—}12214.1), LIMK2 (NP_{—}005560.1), PIP5K1A (AAC50911.1), STK16 (CAA06700.1), LIMK1 (NP_{—}002305.1), DAPK1 (NP_{—}004929.2), PTK2B (NP_{—}775267.1), CDK9 (NP_{—}001252.1), RPS6KA1.Kin.Dom.1 (NP_{—}002944.2), and CLK4 (NP_{—}065717.1).

8. The method of claim 6, wherein said test compound is tested at a concentration of about 10 μM.

9. The method of claim 6, wherein step b) comprises determining the ability of the compound to inhibit the kinase activity of at least twelve kinases selected from said group.

10. The method of claim 9, wherein step b) comprises determining the ability of the compound to inhibit the kinase activity of all primary kinases in said group.

11. A method for screening compounds for potential genotoxicity, said method comprising:
a) providing a plurality of test compounds;
b) determining the ability of each compound to inhibit the kinase activity of at least ten kinases selected from the group consisting of CAMK2A (NP_{—}741960.1), CAMK2D (AAD20442.1), DYRK1B (NP_{—}004705.1), MAPK15 (NP_{—}620590.2), PCTK2 (CAA47004.1), PFTK1 (NP_{—}036527.1), PCTK1 (NP_{—}006192.1), PCTK3 (NP_{—}002587.2), CDK2 (cyclin dependent kinase 2, NP_{—}001789.2), GSK3A (NP_{—}063937.2), CDK3 (NP_{—}001249.1), CLK2 (NP_{—}003984.2), MELK (NP_{—}055606.1), BRSK2 (NP_{—}003948.2), CAMK1 (NP_{—}003647.1), STK3 (NP_{—}006272.1), MYLK (NP_{—}444254.3), CDK5 (NP_{—}004926.1), FLT3 (NP_{—}004110.2), FLT3.ITD (NP_{—}004110.2), PRKR (NP_{—}002750.1), and AMPKα2 (NP_{—}006243.2);
wherein inhibition of at least five of said kinases by 100% indicates a likelihood that said test compound will demonstrate genotoxicity.

12. The method of claim 11, further comprising:
c) rejecting compounds that demonstrate a likelihood of genotoxicity.

13. The method of claim 1, wherein the ability of the compound to inhibit the kinase activity is determined by measuring the binding affinity of the compound for said kinases.

14. A test substrate, comprising:
A solid support; and
Immobilized on said solid support, the kinases CAMK2A (NP_{—}741960.1), CAMK2D (AAD20442.1), DYRK1B (NP_{—}004705.1), MAPK15 (NP_{—}620590.2), PCTK2 (CAA47004.1), PFTK1 (NP_{—}036527.1), PCTK1 (NP_{—}006192.1), PCTK3 (NP_{—}002587.2), CDK2 (cyclin dependent kinase 2, NP_{—}001789.2), GSK3A (NP_{—}063937.2), CDK3 (NP_{—}001249.1), CLK2 (NP_{—}003984.2), MELK (NP_{—}055606.1), BRSK2 (NP_{—}003948.2), CAMK1 (NP_{—}003647.1), STK3 (NP_{—}006272.1), MYLK (NP_{—}444254.3), CDK5 (NP_{—}004926.1), FLT3 (NP_{—}004110.2), FLT3.ITD (NP_{—}004110.2), PRKR (NP_{—}002750.1), and AMPKα2 (NP_{—}006243.2).

15. The test substrate of claim 14, further comprising:
Immobilized on said solid support, a kinase selected from the group consisting of SLK (NP_{—}055535.2), NUAK1 (NP_{—}055655.1), CAMKK2 (NP_{—}006540.3), BRSK1 (NP_{—}115806.1), GSK3B (NP_{—}002084.2), TTK (NP_{—}003309.2), CAMK2G (NP_{—}751913.1), ALK (NP_{—}004295.2), AAK1 (NP_{—}055726.3), ACVR2A (NP_{—}001607.1), CLK1 (AAA61480.1), BIKE (NP_{—}060063.2), SNARK (NP_{—}112214.1), LIMK2 (NP_{—}005560.1), PIP5K1A (AAC50911.1), STK16 (CAA06700.1), LIMK1 (NP_{—}002305.1), DAPK1 (NP_{—}004929.2), PTK2B (NP_{—}775267.1), CDK9 (NP_{—}001252.1), RPS6KA1.Kin.Dom.1 (NP_{—}002944.2), and CLK4 (NP_{—}065717.1).
